(19) United States
(12) Patent Application Publication
(10) Pub. No.: US 2022/0133586 A1
(43) Pub. Date: May 5, 2022

(54) HANDHELD BABY MASSAGE DEVICE

(71) Applicant: Elina Furman, Dobbs Ferry, NY (US)

(72) Inventor: Elina Furman, Dobbs Ferry, NY (US)

(21) Appl. No.: 17/570,214

(22) Filed: Jan. 6, 2022

Related U.S. Application Data
(60) Provisional application No. 63/134,476, filed on Jan. 6, 2021.

(51) Int. Cl. A61H 23/02

(52) U.S. Cl. CPC A61H 23/0254; A61H 2201/1215; A61H 2201/1685; A61H 2201/0157; A61H 2201/1695; A61H 2201/0153

(57) ABSTRACT

Interactive massage tool for babies/toddlers. The baby massage device/tool can have multiple vibration settings identified by the inventor as ideal for baby massage and growth. The massage device can have multiple interchangeable massage heads including a smooth head, an acupressure head, and a head including bristles for bath time massage and skin sensation stimulation. The baby massage device can have a simple single button operation. By promoting the bonding and nurturing aspects of infant/baby massage with the accessible baby message devices and methods disclosed herein and an educational content platform, the inventor hopes to raise awareness of the innumerable physical and psychological benefits of infant massage for baby and their caretakers. In creating a safe, waterproof baby massager using only the most premium medical-grade materials, the inventor hopes to inspire and educate parents on the critical, scientifically backed practice of infant massage that is widely used all over the world.

### CROSS-REFERENCE TO RELATED APPLICATIONS

This application claims priority to and the benefit of U.S. Provisional Application No. 63/124,476 filed Jan. 6, 2021, the contents of which are hereby incorporated by reference herein in its entirety and for all purposes.

### BACKGROUND OF THE INVENTION

As a mom and baby product expert, the inventor worked and consulted with dozens of baby product companies. Having suffered from post-partum anxiety with the inventor's first-born son who had colic issues, the inventor was inspired to create the baby massage device and methods disclosed herein to help educate and bring awareness to infant massage. The inventor regretted not getting trained or learning about infant massage earlier in her motherhood journey and wished she could go back in time to perform massage on both of her sons during their key developmental years. The inventor had heard so much about the importance of nursing, attachment parenting, baby sign language, skin-to-skin contact but almost nothing about the proven, evidence-based benefits of infant massage for moms, fathers, and baby, especially for those mothers struggling with postpartum issues and bonding in the fourth trimester and beyond.

With the inventor's oldest suffering from anxiety today, the inventor believes that early intervention could have helped him regulate his stress levels and help her alleviate much of the maternal anxiety she felt during the 0-1 year developmental mark. The inventor wished she had known about the role that touch and massage plays in establishing the connection between mom and baby.

After many years in the baby industry, the inventor had seen many products come and go but none that she felt made a huge difference to baby's neural, emotional, and cognitive development. The inventor also felt that many of the products in today's baby market emphasized separation of baby and caretaker using cribs and swings. The inventor wanted to create something that gave parents a way to improve their connection and increase their interaction with their baby.

After talking to many parents, the inventor found that many were confused and bewildered about infant massage and were worried that they were doing it wrong or that they needed formal training before performing massage on their own babies.

That's when the inventor had the idea to create the first soothing and bonding interactive massage tool tailored to the unique needs of mother/caretaker and child. Using soft percussion and massage, the inventor's goal was to create something safe, low-vibrational, and specifically customized to baby's smaller anatomical scale. As a CIMI (Certified Infant Massage Instructor), the inventor is uniquely trained to teach parents how to massage and sooth baby.

By helping to empower and educate families about the importance of two-way physical communication in an ever increasingly disconnected world, the inventor hopes to provide a strong foundation for families built on trust, touch, and love.

For parents looking for a method to promote growth, healing, and bonding with their baby while also helping relieve many of the maternal mental health issues that can crop up in the 4th trimester, it's important to consider just how beneficial a daily massage routine could be for both parent and baby.

Every parent wants to bond with their newborn child, while also promoting their overall health and development. There are many tips and tricks to parenting, but few are as incredible or natural as baby massage.

In India and other Eastern countries, baby massage is considered one of the most important aspects of baby rearing with many families hiring “Ammas,” women who come to the house to perform massage, on a daily basis.

### Baby Massage Studies

### Physical, Cognitive, Emotional Benefits

Many studies have established many physical, cognitive, and emotional benefits of baby massage. A short summary of just a few such studies follows as examples.

In a study of 40 infants born early, those massaged three times a day for 15 minutes each time showed significantly more weight gain starting on the fourth day, and that was followed by earlier discharge. Massage therapy can promote weight gain in very low birth weight neonates and also leads to earlier discharge. Rad Z A, Haghshenas M, Javadian Y, Hajiahmadi M, Kazemian F. The effect of massage on weight gain in very low birth weight neonates. J Clin Neonatol 2016; 5:96-9.

In another study, massage on healthy, preterm infants showed a positive influence on brain and visual development. Preterm infants receiving massage therapy gained more weight and slept less. Massage therapy led to weight gain and increased bone density in preterm infants. Preterm Infant Massage Therapy Research: A Review, T. Field, M. Diego, M, Hernandez-Reif, Infant Behav Dev., 2010 Apr. 3: 115-124.

Massage combined with physical activity improved bone mineralization in premature infants. Physical Activity Combined with Massage Improves Bone Mineralization in Premature Infants: A Randomized Trial, H. Aly, M. Moustafa, S. Hassanein, A Massaaro, H. Amer, K. Paatel; Journal of Perinatology 2004; 24:305-309.

### Maternal Health

The infants who experienced massage therapy compared to infants in the rocking control group spent more time in active alert and active awake states, cried less, and had lower cortisol levels, suggesting lower stress. Over the 6-week period, the massage-therapy infants gained more weight, showed greater improvement on emotionality, sociability, and soothability temperament dimensions, and had greater decreases in stress neurotransmitters/hormones (norepinephrine, epinephrine, and cortisol). Field, T., Grizzle, N., Scafidi, F. Abrams, S., Richardson, S., Kuhn, C., & Schanberg, S.; Massage therapy for infants of depressed mothers; Infant Behavior and Development, 19, 107-112 (1996).

Mothers of preterm infants had lower levels of depression and anxiety after massaging their infants. Mothers' depressed mood and anxiety levels are reduced after massaging their preterm infants; L. Feijo; M. Reif, T. Field, W Burns, S. Valley-Gray, E. Simco; Infant Behavior & Development 29 (2006) 476-480.

### Sleep More, Cry Less

Infants and toddlers with sleep onset problems were given daily massages by their parents for 15 minutes prior to bedtime for one month. Based on parent diaries the massaged versus the control children (who were read bedtime stories) showed fewer sleep delay behaviors and had a shorter latency to sleep onset by the end of the study. Forty-five-minute behavior observations by an independent observer also revealed more time awake, alert and active and more positive affect in the massaged children by the end of the study. T. Field, M. Hernandez-Reif; Early Child Development and Care, 168, 95-104 (2001).

### Paternal Bonding

Infants were given massages by their fathers for 15 minutes prior to their daily bedtime for one month. By the end of the study, the fathers who massaged their infants were more expressive and showed more enjoyment and more warmth during floor-play interactions with their infants. C. Cullen, T. Field, A. Escalona, A. & Hartshorn, K.; Father-infant interactions are enhanced by massage therapy; Early Child Development and Care, 164, 41-47 (2000).

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY OF THE INVENTION

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

Baby massage devices, methods of use, and methods of manufacture are disclosed. The baby massage device can include a silicon handle sized and shaped for being held by a baby as well as an adult caregiver. The baby massage device can include an interchangeable massage head made of silicone for providing a vibratory massage to the baby. The baby massage device can include a vibrating motor disposed within a cap connected to a distal end of the handle, the vibrating motor. The baby massage device can include control circuitry configured to control the vibrating motor for providing a massage vibration to the interchangeable massage head.

The handle can include an outer handle surface comprising an anti-microbial, medical grade silicone for hygiene and baby's safety.

The baby massage device can have a single button operation to toggle between multiple vibrational settings.

The baby massage device can include multiple interchangeable massage heads, the multiple interchangeable massage heads include a first smooth head, a second acupressure head having massage protrusions, and a third silica bath head with bristles.

The control circuit can include a first vibration setting between 10-11 cycles per second, a second vibration setting between 13-14 cycles per second, and a third vibration setting between 16-17 cycles per second. According to a preferred embodiment, the control circuit has a first vibration setting of 10.6 cycles per second for a first stage of 1-6 months, a second vibration setting of 13.6 cycles per second for a second baby stage of 6-12 months, and a third vibration setting of 16.6 cycles per second for a third baby stage of 12+months.

The vibrating motor preferably makes less than 50 decibels of noise in operation.

The baby massage device is preferably at least IPX6 waterproof rating for use during bathing the baby.

The handle can include a relatively thin diameter of the handle proximate to the head interchangeable head about 20 millimeters thick in diameter. The handle can include a relatively wider portion at least 35 millimeters in diameter such that both the baby with relatively small hands and the caretaker with relatively large hands can handle the baby massage device.

The baby massage device can include a bottom cap, the bottom cap including three acupressure foot massage protrusions sized and configured for small finger/hand and toe/feet massage.

The baby massage device can include a first handle case and a second handle case assembled with internal components disposed therein, the internal components including a printed circuit board including the control circuitry and a battery. The baby massage device can further include a first cap and a second cap assembled for locating and enclosing the vibration motor therein. The baby massage device can further include a silicone cover covering the assembled caps and providing a silicone cover thereover. The silicone handle covering the assembled handle cases.

The baby massage device can have a length of the baby massage device from a top of the interchangeable massage head to a bottom of the massage protrusions of the bottom cap is less than 17 centimeters. The length of the baby massage device from the top of the interchangeable massage head to the bottom of the massage protrusions of the bottom cap can be less than 16 centimeters.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIGS. 1 and 2 illustrate a baby massage device with interchangeable massage heads according to an embodiment of the invention;

FIGS. 3 and 4 illustrate use of the baby massage device for soothing a baby along with additional massage techniques;

FIGS. 5 and 6 illustrate exploded views of the baby massage device; and

FIG. 7 illustrates a Z axis cross-sectional view of the baby massage device.

### DETAILED DESCRIPTION OF THE PREFERRED EMBODIMENTS

Embodiments of the invention disclosed herein relate to the first, and multiple award winning, interactive massage tool for babies/toddlers. By promoting the bonding and nurturing aspects of infant/baby massage with the accessible message devices and methods disclosed herein and an educational content platform, the inventor of this patent application hopes to raise awareness of the innumerable physical and psychological benefits of infant massage for baby and their caretakers.

In creating a safe, waterproof baby massager using only the most premium medical-grade materials, the inventor hopes to inspire and educate parents on the critical, scientifically backed practice of infant massage that is widely used all over the world.

The inventor is a well-known baby product expert with over 15 years of experience consulting on and marketing baby products. She has been featured as a baby product expert on the Today Show, CNN and as a featured judge/baby division at the New York International Gift Fair. She is also an advisor for Gugu Guru, a top baby registry site.

The inventor is a certified Infant Massage Instructor who has spent over a year developing the baby massage device disclose herein. She believes strongly that infant massage is one of the most natural and underutilized practices that once adopted in the US as a common mainstream practice will have innumerable benefits for babies' well-being and development and enhancing the bond between caretaker and child.

By creating a safe, waterproof baby massage device using only the most premium medical-grade materials, the inventor hopes to inspire and educate parents on the critical, scientifically backed practice of infant massage that is widely used all over the world.

For babies, the benefits of using such baby massage devices and methods include promoting social, emotional, and cognitive development, helping the baby relax and release tensions of daily stimuli, decreased irritability and reduction of excessive crying, reduction of gas, colic and intestinal difficulties, regulating behavioral states and promoting sleep, strengthening and regulating primary systems (i.e., respiratory, circulatory, nervous, musculature, digestive and endocrine).

For the mother, the benefits of using such baby massage devices and methods include promoting better understanding of infant cues, enhances communication and emotional ties to baby, increasing confidence and handling skills, providing quality one-on-one interaction, encouraging parents to unwind, relax, and listen to their baby.

As discovered by the inventor, having a physical baby massage tool creates intention and awareness, promotes consistency of use. Ease of use using such baby massage devices disclosed herein builds confidence in caretakers, forms a habitual behavior. Moreover regular, daily massage creates new neural pathways for mom and baby forming a habit of using a tool allows caregiver to relax and destress daily. Portability of the baby massager reminds mom to perform massage on the go.

The invention disclosed herein is the first product to combine massage and the calming/therapeutic effects of vibration into one package with the added benefit of increasing interaction between child/caretaker that is so vital to baby's psychosocial and emotional development.

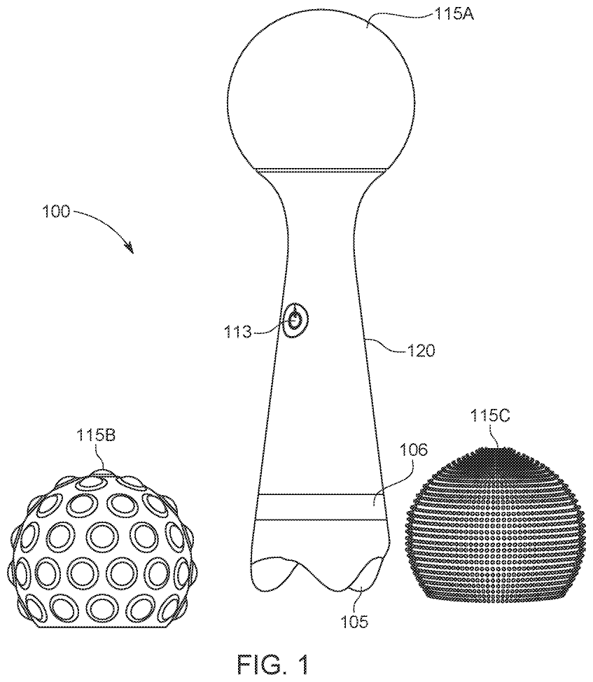

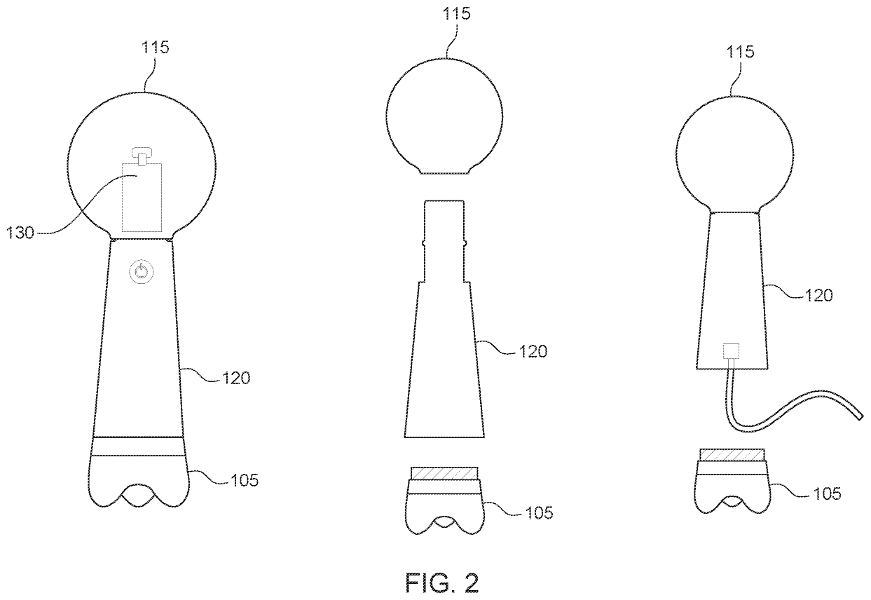

Referring to FIG. 1, a baby massage device 100 is illustrated according to an example of the present invention. The baby massage device 100 can include a silicone handle 120. As disclosed herein the silicone components can include an anti-microbial, medical grade silicone for superior hygiene and baby's safety. The silicone can be edible grade if baby places in mouth.

The baby massage device 100 can have a single button 113 operation to toggle between multiple vibrational settings.

The baby massage device 100 can further include multiple interchangeable heads 115. As illustrates, the multiple interchangeable heads 115 can include a first smooth soothing head 115A, a second acupressure soft touch head 115B, and a bath time head 115C as has been discovered to be particularly advantageous by the inventor. The smooth head 115A can be used for daily massage including calming and relaxing the baby. The second acupressure head 115B can be used for deeper stimulation, energizing, and reflexology for baby. The third silica bath head 115C can be used as a bath massager and scalp stimulator with bristles designed to specific length for baby's safety.

The acupressure head 115B features soft touch technology that are used as a safe way to help calm or ease pain and help babies maintain a healthy immune and digestive system.

The bath time head 115C keeps baby's biggest organ, their skin, flake-free and soft and aids with cradle cap, gently removing dry skin from scalp.

The baby massage device 100 further includes a vibrating motor 130. The inventor spent a lot of time working on the vibration by the motor 130 and distributed as well as focused on the head 1115 to make sure the vibration was low-vibration but still impactful. The inventor discovered that it's not just about the level of vibration but also how it's distributed throughout the unit, with most of the vibrations being in the head of the unit rather than in the handle.

When it comes to vibration, the cycles are the most important factor since they can range from very low to very high which wouldn't be appropriate for babies. According to advantageous designs disclosed herein, the vibration settings can have a first vibration setting at 10-11 cycles per second, a second vibration setting at 13-14 cycles per second, and a third vibration setting at 16-17 cycles per second.

According to a preferred embodiment, the baby massager 100 can have a first vibration setting of 10.6 cycles per second for a first stage of 1-6 months, a second vibration setting of 13.6 cycles per second for a second baby stage of 6-12 months and a third vibration setting of 16.6 cycles per second for a third baby stage of 12+ months.

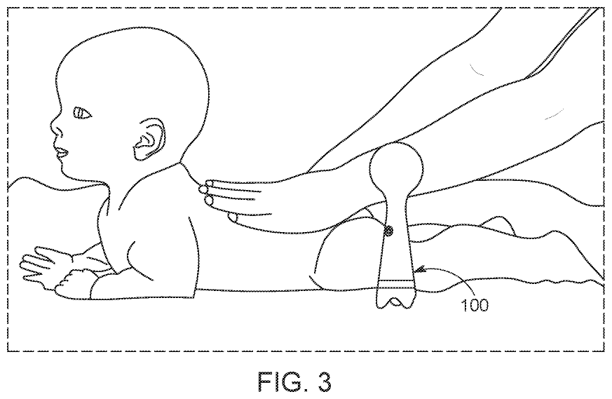

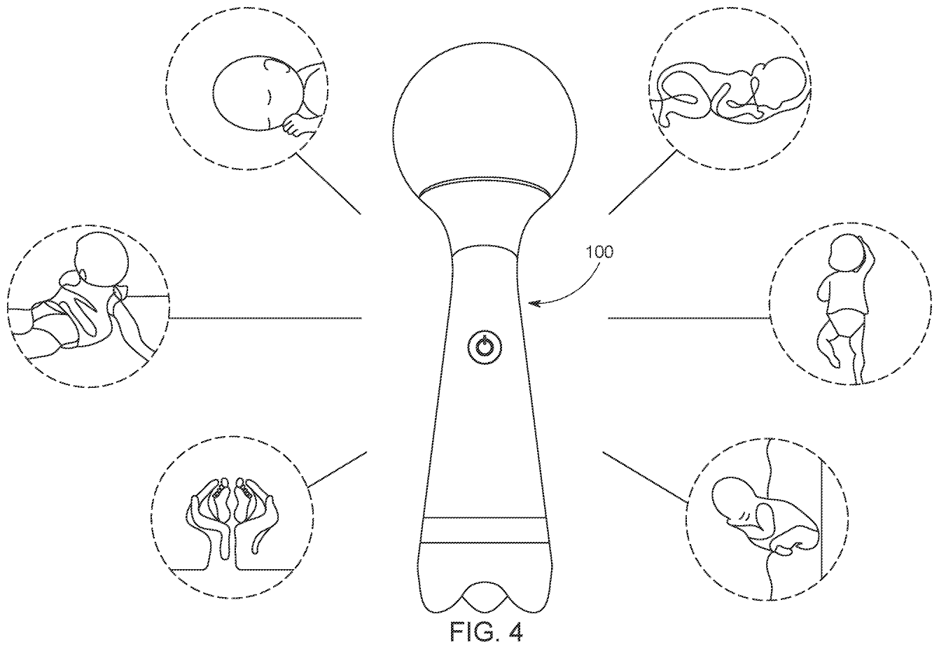

The motor 130 is preferably whisper quiet operation (below 50 decibels) so as to sooth and relax the baby as seen in FIGS. 3 and 4.

The baby massage device is preferably at least IPX6 waterproof rating such that the baby massage device 100 is safe to be submerged during bath time.

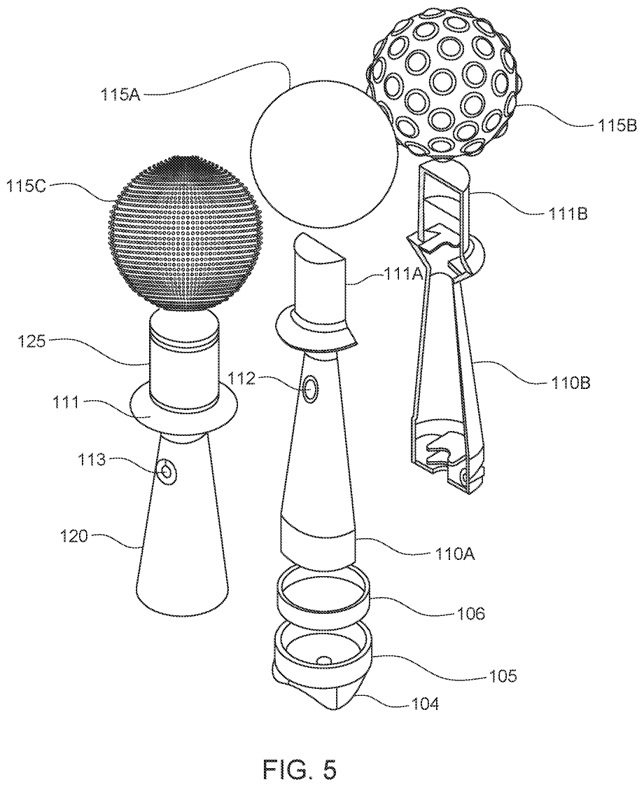

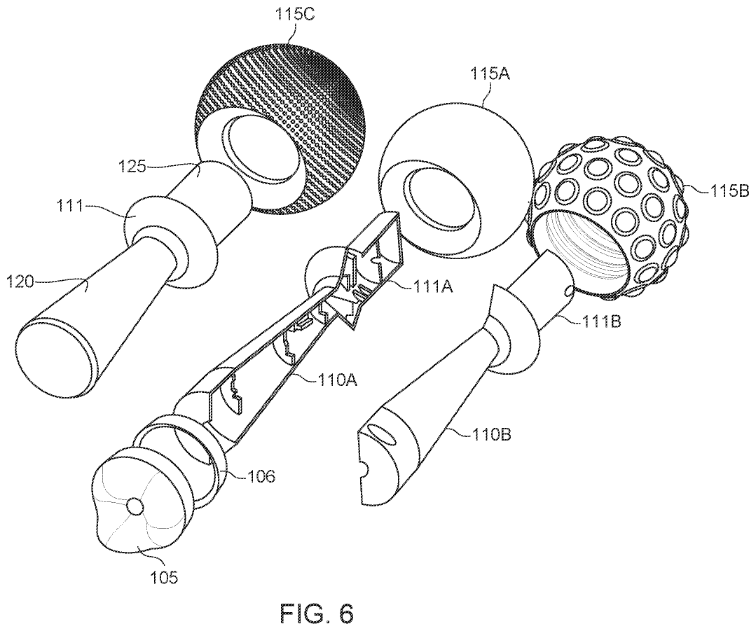

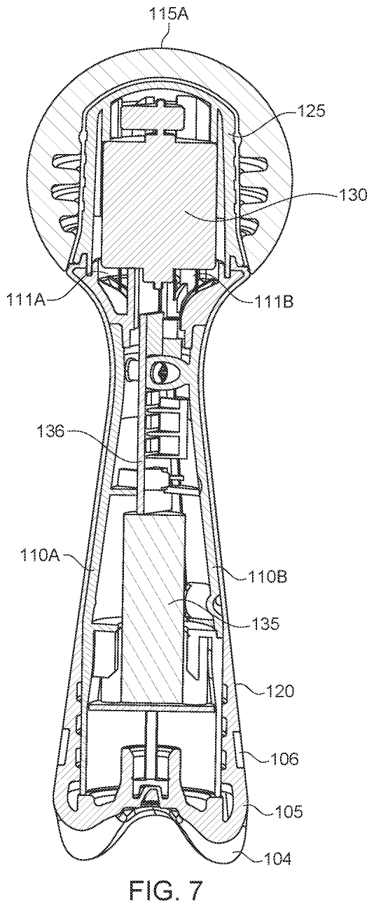

Referring to FIGS. 5 and 6 two exploded views of the baby massage device 100 are illustration. Referring also to FIG. 7, a Z axis cross-section view of the baby massage device 100 is illustrated. The illustrations of FIGS. 5-7 illustrate the components of the baby massage device 100 and examples of methods of manufacture and assembly of the baby massage device according to an example embodiment thereof.

The baby massage device can have a first handle case 110A and a second handle case 110B. The handle cases 110 can be made of ABS injection molding process, for example and assembled together with the internal components illustrated in FIG. 7.

The baby massage device 100 can further include a first cap 111A and a second cap 111B assembleable together for locating and enclosing the motor 130 therein.

The baby massage device 100 can further include a silicone cover 125 for covering the assembled caps 111 and providing a silicone cover thereover.

The baby massage device can further include the silicone handle 120 configured to cover the assembled handle cases 110.

The baby massage device can further include a metal ring 106 and a screw off bottom cap 105. The screw off bottom cap 105 can include three molded massage protrusions as shown particularly sized and shaped for massaging the baby. The length of the prosecutions can be between 3 and 9 millimeters in length, for example. According to preferred embodiments, the protrusions can be about 6 millimeters in length and about 2 centimeters in dimeter The size and diameter of the handle 120 can be particularly critical such that is enables ii both the baby and the caretaker to hold and use the baby massage device 100. For example, relatively thin diameter of the handle 120 proximate to the heads 115 can be about 20 millimeters thick in diameter. Whereas a relatively wider portion of the baby massage device 100 can be at least 30-35 millimeters such that both the baby with relatively small hands and the caretaker with relatively large hands can handle the baby massage device 100.

According to preferred embodiments, the total length of the baby massage device 100 from the top of the massage head 115 to the bottom of the massage protrusions 104 of the bottom cap 104 is less than 17 centimeters, preferable about, or less than, 16 centimeters.

Referring to FIG. 7, the baby massage device 100 can include a battery 135 and printed circuit board (PCB) 136 including control circuitry for providing power to and control of the motor 130 as disclosed herein. The battery 135 can be a Lithium-ion battery, which can be rechargeable for 60 hours of operation, for example.

The baby massage device 100 is ergonomically designed to fit baby's anatomy and parent's hands. The size specifications of the baby massage device 100 are designed to be used with baby's smaller scale and anatomy. The handle 120 has an easy grasp neck for baby's small grasp to enhance self-soothing.

The bottom cap has the 3 small acupressure foot massage protrusions 104 sized and configures for small finger/hand and toe/feet massage. A groove between head and body is designed to allow for kneading and gliding massage motion recommended for baby's arms and legs.

The smaller scale of the baby massager ensures easy portability to calm baby on the go and use as distraction during doctor visits and routine trips.

As discovered by the inventor there are nine top benefits of baby massage, which can be implemented, improved, and benefited using the devices and methods disclosed herein.

### Bonding

A University of Rochester study showed that nearly 35% of new parents in the US do not know what to expect from their newborns, or how to help them grow. Bonding doesn't happen naturally for every parent. But massage using the baby massage device 100, for example, can help. With each massage stroke, a caretaker is showing baby that they love them and care for them and it allows for a deeper connection to grow. Baby massage using the baby massage device 100 also helps parents feel more competent and teaches their baby that they are a trusted and stabilizing force in their life. Ultimately, it will promote a relaxed and safe environment between the two of you which is the key to bonding.

### Digestion

Baby constipation, colic, reflux and gas are some of the most dreaded aspects of new parenthood.

At birth, a baby's digestive tract is very underdeveloped and often needs a natural kick start with the aid of tummy massage and foot reflexology using the baby massage device 100, among other massage methods for example, has been found to be advantageous for such massage. Both are very easy to do. Just start by paddling baby's tummy and do circles in a clockwise motion. For feet, concentrate on the center of the foot with the baby massage device and make little circles to help the smaller and bigger intestines do their work. Just make sure to wait 30 minutes after feeding.

### Brain and Motor Development

One of the reason parents don't do baby massage more often is that they are not aware of all the cognitive benefits of massage. In fact, 23% of parents didn't know it had any brain boosting benefits at all according to a recent study. Another incredible benefit of baby massage is that it can actually improve their motor development. Since massage is such a powerful sensory experience, it actually stimulates the brain which helps children develop their fine motor skills as they grow up. In particular, massage using the baby massage device 100, for example, can help with motor development if a child has a developmental delay or disability, as evidenced by studies in the National Library of Medicine.

### Sleep

When it comes to babies and sleep, parents will try everything. That's why it's a big surprise that massage is not the first go-to in a mama's toolbox. One of the best ways to encourage healthy sleep patterns is to create a serene bedtime routine. The inventor has recommended giving babies a massage using the baby massage device 100, for example, right after bath and before feeding. Parents should give them the massage while they are still awake and alert, but make sure they are not overstimulated or fussy. Newborns love and anticipate routine. Help them relax prior to their bedtime by creating a calming environment using dim lighting and soft music.

### Body Awareness

Proprioception is our sense of our bodies and how we move through space. When babies are little, they have little to no awareness of their fingers, thumbs or feet. Even finding their thumb to suck is a challenge. When performing infant massage using the baby massage device, baby is getting an opportunity to process many different sensory inputs such as touch and pressure which helps them make those important neural connections and will help them develop a strong sense of body awareness which contribute to reaching milestones faster.

### Immunity

Life with baby during a pandemic is no easy feat. Once you add in cold and flu season, the last thing any parent wants is for baby to catch a virus. The wonderful thing about baby massage using the easily cleaned and safe baby massage device 100, for example, is that it can actually help boost baby's immunity. During massage using the baby massage device, parents are draining the lymphatic system and flushing out the toxins in baby's system. It's an immunity boost that is sorely needed during this time.

### Weight Gain

If a baby is having difficulty putting on weight and a parent is worried about their weight, consider the research. Tiffany Fields conducted research that found preemies who were massaged gained significantly more weight than those that weren't. During the massage using the baby massage device 100, a parent is actually stimulating what is called the Vagus nerve which helps baby's feed and nurse more effectively which can aid in weight gain for a newborn.

### Maternal and Paternal Wellbeing

While some mamas and papas can breeze through the post-partum period unscathed, many will experience symptoms of baby blues, depression or anxiety. Infant massage using the baby massage device, for example, isn't only important for a newborn baby, but also an excellent way for you to create a calming routine for a child so that parents are also at their best. By taking time to calm down and connect with baby, the caretaker find that they also feel the release of those feel-good hormones like Oxytocin. Many parents also report that they feel more competent in handling their babies after consistently performing baby massage which leads to greater confidence and well-being. Creating a space to relax, recharge, and bond with your child in these early developmental years is crucial.

It is understood that when an element is referred hereinabove as being “on” another element, it can be directly on the other element or intervening elements may be present there between. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A baby massage device comprising:
a handle sized and shaped for being held by a baby as well as an adult caregiver;
an interchangeable massage head made of silicone for providing a vibratory massage to the baby;
a vibrating motor disposed within a cap connected to a distal end of the handle, the vibrating motor;
control circuitry configured to control the vibrating motor for providing a massage vibration to the interchangeable massage head.

2. The baby massage device according to claim 1, wherein the handle includes an outer handle surface comprising an anti-microbial, medical grade silicone for hygiene and baby's safety.

3. The baby massage device according to claim 1, wherein the baby massage device has a single button operation to toggle between multiple vibrational settings.

4. The baby massage device according to claim 1, further include multiple interchangeable massage heads, the multiple interchangeable massage heads include a first smooth head, a second acupressure head having massage protrusions, and a third silica bath head with bristles.

5. The baby massage device according to claim 1, wherein the control circuitry includes a first vibration setting between 10-11 cycles per second, a second vibration setting between 13-14 cycles per second, and a third vibration setting between 16-17 cycles per second.

6. The baby massage device according to claim 1, wherein the control circuitry has a first vibration setting of 10.6 cycles per second for a first stage of 1-6 months, a second vibration setting of 13.6 cycles per second for a second baby stage of 6-12 months, and a third vibration setting of 16.6 cycles per second for a third baby stage of 12+ months.

7. The baby massage device according to claim 1, wherein vibrating motor makes less then 50 decibels of noise in operation.

8. The baby massage device according to claim 1, wherein the baby massage device at least IPX6 waterproof rating.

9. The baby massage device according to claim 1, wherein the handle includes a relatively thin diameter of the handle proximate to the head interchangeable head about 20 millimeters thick in diameter.

10. The baby massage device according to claim 9, wherein the handle includes a relatively wider portion at least 35 millimeters in diameter such that both the baby with relatively small hands and the caretaker with relatively large hands can handle the baby massage device.

11. The baby massage device according to claim 1, further comprising a bottom cap, the bottom cap including three acupressure foot massage protrusions sized and configured for small finger/hand and toe/feet massage.

12. The baby massage device according to claim 1, further comprising a first handle case and a second handle case assembled with internal components disposed therein, the internal components including a printed circuit board including the control circuitry and a battery.

13. The baby massage device according to claim 12, further comprising a first cap and a second cap assembled together for locating and enclosing the vibration motor therein.

14. The baby massage device according to claim 13, further comprising a silicone cover covering the assembled caps and providing a silicone cover thereover.

15. The baby massage device according to claim 14, the silicone handle covering the assembled handle cases.

16. The baby massage device according to claim 15, further comprising a screw off bottom cap, the screw off bottom cap including three molded massage protrusions.

17. The baby massage device according to claim 16, wherein the silicone handle includes a thin diameter of the silicone handle proximate to the interchangeable head about 20 millimeters thick in diameter, the silicone handle further includes a relatively wider portion at least 35 millimeters in diameter.

18. The baby massage device according to claim 17, wherein a length of the baby massage device from a top of the interchangeable massage head to a bottom of the massage protrusions of the bottom cap is less than 17 centimeters

19. The baby massage device according to claim 18, wherein the length of the baby massage device from the top of the interchangeable massage head to the bottom of the massage protrusions of the bottom cap is less than 16 centimeters.

20. The baby massage device according to claim 19, further comprising multiple interchangeable massage heads, the multiple interchangeable massage heads include a first smooth head, a second acupressure head having massage protrusions, and a third silica bath head with bristles.
